(19) United States
(12) Patent Application Publication
(10) Pub. No.: US 2014/0187638 A1
(43) Pub. Date: Jul. 3, 2014

(54) PHENYLBUTYRATE IN RECTAL FORM FOR THE TREATMENT OF A MOTOR NEURON DISEASE OR A METABOLIC DISEASE

(71) Applicant: Frederic Marin, Paris (FR)

(72) Inventor: Frederic Marin, Paris (FR)

(73) Assignee: GMP-ORPHAN, Paris (FR)

(21) Appl. No.: 14/128,346

(22) PCT Filed: Jun. 22, 2012
(86) PCT No.: PCT/EP2012/062152

§ 371 Date: Dec. 20, 2013

Related U.S. Application Data
(60) Provisional application No. 61/499,805, filed on Jun. 22, 2011.

(51) Int. Cl. A61K 9/00; A61K 31/192

(52) U.S. Cl. CPC A61K 9/0031; A61K 31/192
USPC 514/576; 562/496

(57) ABSTRACT

The present invention relates to rectal administration of phenylbutyrate for treating a motor neuron disease, such as, for example, spinal muscular atrophy or a metabolic disease such as, for example, an urea cycle disorder, related methods and compositions.

### FIELD OF INVENTION

The present invention relates to a composition comprising a therapeutically acceptable salt of 4-phenylbutyrate in a rectal formulation for the treatment of a motor neuron disease, such as, for example, spinal muscular atrophy; or of metabolic diseases, such as, for example, urea cycle disorder or of cancer. The present invention further relates to a method for treating a motor neuron disease, such as, for example, spinal muscular atrophy; or metabolic diseases, such as, for example, urea cycle disorder or cancer; said method comprising rectal administration of a therapeutically acceptable salt of 4-phenylbutyrate.

### BACKGROUND OF INVENTION

Spinal muscular atrophy (SMA) is rare genetic orphan disease (one in 6000 to 10000 births) characterized by the progressive degeneration of motoneurons and by the muscle weakness and atrophy. Locomotion, body posture, respiration and deglutition are progressively disrupted, while cognition is unaffected. SMA is the leading genetically inherited cause of death of children under the age of two years.

Four types of spinal muscular atrophy affect children before the age of 1:
Type I spinal muscular atrophy, also called Werdnig-Hoffman disease, is a severe form of the disorder that is evident at birth or within the first few months of life. Typically, affected infants have difficulty breathing and swallowing and are unable to sit without support.
Type II spinal muscular atrophy is characterized by muscle weakness that develops in children between ages 6 and 12 months. Children with type II can sit without support, although they cannot stand or walk unaided.
X-linked infantile spinal muscular atrophy has features that are very similar to type I, except that children with this type are typically born with joint deformities that impair movements.
The fourth type of spinal muscular atrophy that appears in infancy is called distal spinal muscular atrophy type I. This form of the disorder is characterized by progressive muscle weakness in the hands and feet that eventually spreads to the limbs.

Three other types of SMA can affect people in early childhood and adulthood: type III (or Kugelberg-Welander disease or juvenile type), type IV and Finkel type, the last two usually occurring after the age of 30.

SMA is an autosomal recessive condition and is mainly caused by mutation in the Survival Motor Neuron (SMN1) gene present on chromosome 5. About 95% of individuals with SMA have mutations that delete a section called exon 7 in both copies of SMN1 gene. This gene encodes the SMN protein which is found throughout the body, with high level in the spinal cord. This protein is important for the maintenance of the specialized nerve cells called motoneurons, which are located in the spinal cord and in the part of the brain that is connected to the spinal cord. The absence of the SMN protein induces an important death of motoneurons and results in SMA symptoms.

The SMN protein may also be produced by the SMN2 gene that is a “backup” copy of the SMN1 gene. However, SMN2 gene mostly encodes for an incomplete and not functional form of SMN protein and produces only low levels of full-length and functional SMN protein. All patients have at least one, generally two to four copies of SMN2 gene in cells, and there is an important variability between people. Therefore, depending on their number of SMN2 gene copies, patient affected by SMA may have more or less severe symptoms.

Current research for the treatment of SMA is mostly oriented toward motoneurons protection or genetic therapies. It yielded in promising results, but the research efforts still require a relatively long development before effective treatment are available to patients.

Another therapeutic strategy in the treatment of SMA consists in increasing transcription of the remaining SMN2 gene, leading to the up-regulation of SMN protein. Increasing gene transcription may be achieved using inhibitor of histone deacetylase (HDAC). Indeed, transcription of DNA is dictated by its structure and especially by histone acetylation. Deacetylation of histones by histone deacetylase implies gene silencing. Therefore, inhibiting this enzyme avoids deacetylation and increases transcription of the gene and therefore production of the corresponding protein.

Some small molecules were tested as HDAC inhibitors, such as valproic acid, hydroxyurea and sodium 4-phenylbutyrate (phenylbutyrate). These compounds are already known for other indications: valproic acid is used for the treatment of epilepsy in oral or injectable form, hydroxyurea is indicated in the treatment of leukemia in oral form and phenyl butyrate is employed in the treatment of urea cycle disorders in oral form, either in tablets or in powder.

Valproic acid and hydroxyurea do not seem to have given significant results in terms of efficacy (Weihl et al., Neurology, 2006, 67(3), 500-501; Liang et al., J. Neurol. Sci., 2008, 268(1-2), 87-94).

A first preliminary open label study suggested efficacy of phenylbutyrate administered in an oral form in the treatment of SMA (WO2005/072720, Mercuri et al., Neuromuscular Disorders, 2004, 14, 130-135). This trial involved 10 patients with SMA type II that were treated with oral phenylbutyrate in powder or tablets. The dosage was 500 mg/kg/day, divided in five doses using an intermittent schedule (7 days on and 7 days off). The efficacy was measured for 9 weeks. A significant increase in scores on a motor scale was found and no major side effect was recorded.

However, a 13 week long randomized, double-blind, placebo-controlled trial failed to support earlier findings (Mercuri et al, Neurology, 2007, 68, 51-55). This trial involved 107 patients with SMA, the dosage was 500 mg/kg/day of phenylbutyrate or a placebo, on intermittent regimen (7 days on and 7 days off). Even though phenylbutyrate was well tolerated; this trial was not shown to be effective in these conditions.

Clinical trials were also seriously hampered by unpleasant taste and odor of the phenylbutyrate in its current oral formulation which impacted seriously patient' compliance, all the more as important doses are required (minimum 450 mg/kg/day).

Finally, no cure for SMA is available to date.

Therefore, it is an object of the present invention to provide a method of treatment of SMA with a good efficacy in vivo and administered in a dosage form adapted to young children and to patients with problems of swallowing. Similar problems are also treated with the present invention for patients suffering from motor neuron diseases such as urea cycle disorder.

Even though clinical trials did not previously demonstrate efficacy of phenylbutyrate administered by oral route; the Applicant surprisingly found that rectal administration of phenylbutyrate to patients affected by SMA lead to good results in terms of efficacy and that this dosage form was well tolerated by these patients.

### SUMMARY

One object of the invention is a method for treating a motor neuron disease (such as, for example, spinal muscular atrophy) or metabolic diseases (such as, for example, an urea cycle disorder) or cancer, said method comprising rectal administration of a therapeutically acceptable salt of 4-phenylbutyrate.

In one embodiment of the invention, the therapeutically acceptable salt of 4-phenylbutyrate is selected from the group comprising sodium, potassium, magnesium or calcium salt.

In one embodiment of the invention, the salt is the sodium salt.

In one embodiment of the invention, rectal administration is achieved by the use of a rectal dosage form selected from the group comprising suppositories, rectal capsules, enemas, rectal gels, rectal foams or rectal ointments.

In one embodiment of the invention, the therapeutically acceptable salt of 4-phenylbutyrate is administered at a dose ranging from 1 to 600 mg/kg/day.

In one embodiment of the invention, the therapeutically acceptable salt of 4-phenylbutyrate is administered from 1 to 10 time per day.

In one embodiment of the invention, the therapeutically acceptable salt of 4-phenylbutyrate is administered to children suffering from a motor neuron disease, such as, for example, spinal muscular atrophy; or for a metabolic disease (such as, for example, an urea cycle disorder) or cancer.

In one embodiment of the invention, the therapeutically acceptable salt of 4-phenylbutyrate is administered to 0-5 years children suffering from a motor neuron disease, such as, for example, spinal muscular atrophy; or for a metabolic disease (such as, for example, an urea cycle disorder) or cancer.

In one embodiment of the invention, the therapeutically acceptable salt of 4-phenylbutyrate is administered to infants suffering from a motor neuron disease, such as, for example, spinal muscular atrophy; or from a metabolic disease, such as, for example, an urea cycle disorder; or cancer.

In one embodiment of the invention, the motor neuron disease is selected from the group comprising spinal muscular atrophy, amyotrophic lateral sclerosis, ischemia, thalassemias, cystic fibrosis, and Huntington disease.

In one embodiment of the invention, the metabolic disease is selected from the group comprising urea cycle disorder and maple syrup urine disease.

The present invention also relates to an acceptable salt of 4-phenylbutyrate for treating a motor neuron disease, such as, for example, spinal muscular atrophy; or an urea cycle disorder or cancer, wherein said acceptable salt of 4-phenylbutyrate is rectally administered.

In one embodiment of the invention, the therapeutically acceptable salt of 4-phenylbutyrate is administered at a dose ranging from 1 to 600 mg/kg/day. In another embodiment of the invention, the therapeutically acceptable salt of 4-phenylbutyrate is administered from 1 to 10 time per day.

In one embodiment of the invention, the therapeutically acceptable salt of 4-phenylbutyrate is administered to children suffering from a motor neuron disease, such as, for example, spinal muscular atrophy; or an urea cycle disorder or cancer. In another embodiment of the invention, the therapeutically acceptable salt of 4-phenylbutyrate is administered to 0-5 years children suffering from a motor neuron disease, such as, for example, spinal muscular atrophy; or an urea cycle disorder or cancer. In another embodiment of the invention, the therapeutically acceptable salt of 4-phenylbutyrate is administered to infants suffering from a motor neuron disease, such as, for example, spinal muscular atrophy; or an urea cycle disorder or cancer.

In one embodiment of the invention, the motor neuron disease is selected from the group comprising spinal muscular atrophy, amyotrophic lateral sclerosis, ischemia, thalassemias, cystic fibrosis, and Huntington disease, preferably said motor neuron disease is spinal muscular atrophy.

In one embodiment of the invention, the metabolic disease is selected from the group comprising urea cycle disorder and maple syrup urine disease.

Another object of the invention is a rectal dosage form comprising a therapeutically acceptable salt of 4-phenylbutyrate for rectal administration.

In one embodiment of the invention, the therapeutically acceptable salt of 4-phenylbutyrate is selected from the group comprising sodium, potassium, magnesium or calcium salt, preferably the sodium salt.

In one embodiment of the invention, the form for rectal administration is selected from the group comprising suppositories, rectal capsules, enemas, rectal gels, rectal foams or rectal ointments.

In one embodiment of the invention, the rectal dosage form comprises from 1 to 800 mg of a therapeutically acceptable salt of 4-phenylbutyrate.

In one embodiment of the invention, the rectal dosage form comprises at least one viscosity agent, and a solvent, preferentially water.

In another embodiment, said viscosity agent is polyvinylpyrrolidone, such as, for example, PVP K 90; cellulose derivatives, such as, for example, HPC H, HPC HF, HEC 250 HX and HEC 250 M; gums such as, for example xanthan gum, such as Satiaxane UCX 930; carbopol derivatives, such as, for example, Carbopol 971P.

### DEFINITIONS

In the present invention, the following terms have the following meanings:
“capsule” refers to solid preparation with hard or soft shells of various shapes and capacities, usually containing a single dose of active substance.
“rectal capsule” refers to solid, single-dose soft capsule that may have a lubricating coating. Preferably, rectal capsules may present an elongated shape and a uniform external appearance.
“suppository” refers to solid, single-dose preparation prepared by compression or moulding or any other technique well known from the skilled artisan, that contains one or more active substances dispersed or dissolved in a suitable basis which may be soluble or dispersible in water or may melt at body temperature, and optionally excipients.
“therapeutically effective amount” refers to the amount of a therapeutic agent necessary and sufficient for slowing down or stopping the progression, aggravation, or deterioration of one or more symptoms of the disease, or condition; alleviating the symptoms of the disease or condition; curing the disease or condition.
“Pharmaceutically acceptable excipient”: an excipient that does not produce an adverse, allergic or other untoward reaction when administered to an animal, preferably a human. It includes any and all solvents, dispersion media, coatings, antibacterial and antifungal agents, isotonic and absorption delaying agents and the like. For human administration, preparations should meet general safety and purity, and optionally sterility and pyrogenicity standards as required by FDA Office of Biologics standards.
“About”: preceding a figure means plus or less 10% of the value of said figure.

### DETAILED DESCRIPTION

This invention relates to a method for treating a motor neuron disease, or a metabolic disease, or cancer in a subject, comprising rectal administration of a therapeutically effective amount of a therapeutically acceptable salt of 4-phenylbutyrate to a subject in need thereof.

The inventors have found that rectal administration of phenylbuterate leads to a higher concentration and a longer presence of phenylbuterate and phenylacetate in plasma.

Examples of motor neuron diseases include, but are not limited to, spinal muscular atrophy, amyotrophic lateral sclerosis, ischemia, thalassemias, cystic fibrosis, and Huntington disease.

Examples of metabolic diseases include, but are not limited to, urea cycle disorder (UCD) and maple syrup urine disease (MSUD), such as, for example, a classic neonatal severe form, an intermediate form, an intermittent form, a thiamine-responsible form or an E3-deficient with lactic acidosis form of MSUD.

This invention also relates to a method for treating a histone deacetylase related disease, comprising rectal administration of a therapeutically effective amount of a therapeutically acceptable salt of 4-phenylbutyrate to a subject in need thereof, wherein said subject is preferably a child.

Examples of histone related disease include, but are not limited to spinal muscular atrophy, amyotrophic lateral sclerosis, ischemia, thalassemias, cystic fibrosis, and Huntington disease.

In one embodiment, this invention relates to a method for treating SMA comprising rectal administration of a therapeutically effective amount of a therapeutically acceptable salt of 4-phenylbutyrate to a subject in need thereof.

The Applicant surprisingly found that rectal administration of phenylbutyrate is effective for the treatment of a motor neuron disease, such as, for example, SMA; or for the treatment of a metabolic disease such as, for example, an urea cycle disorder or for the treatment of cancer, not only for stopping the worsening of the condition of patients, but also—more surprisingly—to improve the condition of these patients, which is not the case when phenylbutyrate is administrated by oral route as demonstrated by the clinical trial disclosed in Mercuri et al. (Neurology, 2007, 68, 51-55).

Without willing to be bound to a theory, it is suggested that the rectal administration overcomes potential degradations of phenylbutyrate due to gastric passage and therefore improves its biodisponibility. It may also avoid the first hepatic passage by directly liberating the major proportion of the active drug in the general circulation—i.e. systemic delivery. This is all the more interesting in the case of phenylbutyrate as it is quickly metabolized in liver and kidneys (Piscitelli et al., J Clin. Pharmacol., 1995, 35, 368-373), corresponding to a short half-life (0.77 h) after per os ingestion (Berg et al., Cancer Chemother. Pharmacol., 2001, 47, 385-390; Kasumov et al., Drug Metab. Dispo., 2004, 32, 10-19). The rectal route is also interesting as the drug absorbed from the anal canal enters the systemic circulation directly. A rectal form presents the advantage to be particularly well adapted to infants and young children as well as to patients with problems in deglutition or swallowing. It presents the additional advantage to overcome problems linked to the unpleasant taste and odor of phenylbutyrate that is responsible in a number of cases of the abandonment of the treatment currently orally administered.

Therefore, a rectal formulation presents the multiple advantages to overcome the unpleasant taste and odor of phenylbutyrate, to simplify its usage and to delay hepatic metabolisation.

According to a first embodiment, rectal administration is achieved by a rectal dosage form comprising a therapeutically acceptable salt of 4-phenylbutyrate.

The invention also relates to a composition comprising a therapeutically acceptable salt of phenylbutyrate in a dosage form for rectal administration.

According to one embodiment, the therapeutically acceptable salt of 4-phenylbutyrate is selected from the group comprising salts of alkali metals, salts of alkali earth metal, suitable salts of amino acids or ammonium salts. Specific examples of salts are salts selected from the group comprising sodium, potassium, magnesium or calcium salts. According to a preferred embodiment, the therapeutically acceptable salt of 4-phenylbutyrate is sodium 4-phenylbutyrate.

According to an embodiment, the rectal dosage form is selected from the group comprising suppositories, rectal capsules, rectal creams, rectal emulsions, rectal solutions, rectal suspensions, rectal tampons, enemas, retention enemas, rectal gels, rectal foams or rectal ointments.

According to an embodiment, the rectal dosage form is not a suppository.

According to an embodiment, suppository comprises phenylbutyrate and a fatty base or a water-soluble base. According to an embodiment, the fatty base is selected from the group comprising hard fat, such as Witepsol H12 or Witepsol H15, cocoa butter, laurif oil, theobroma oil, mono-, di- tri-glycerides, fatty acids, glycerol-gelatin bases, hard butter, estarinum, suppocire, macrogols, or agrasup. According to an embodiment, the water-soluble base is selected from the group comprising myrj 51, PEGa or polysorbates such as Tween 61 or Tween 60.

According to an embodiment, rectal capsule comprises phenylbutyrate and a hollow shell. According to one embodiment, phenylbutyrate is present in the rectal capsule as a powder, as a solution or as a suspension. In the case of a solution or a suspension of phenylbutyrate, the pH of the solution or the suspension may range from 1 to 7, preferably from 2 to 6, more preferably from 3 to 5. According to one embodiment, the rectal capsule has a size ranging from number 000 to number 5, preferably ranging from number 0 to number 4, more preferably ranging from number 1 to number 3. According to one embodiment, the hollow shell is hard or soft. According to one embodiment, the hollow shell is made of a material selected from the group comprising hard or soft gelatin, glycerol, sorbitol, pullulane and/or cellulose. According to one embodiment, the rectal capsule is coated with a lubricant such as triglycerides, glycerin or polyglycerin. According to another embodiment, rectal capsule further comprises pharmaceutically acceptable excipients such as diluants, opaque fillers, antimicrobial preservatives, sweeteners, coloring matter, stabilizers, biocompatible polymers, oils, surfactants, dispersants or water absorbents. Selection of excipients and optimization of the concentration thereof are well within the ability of the skilled artisan.

According to an embodiment, enema comprises phenylbutyrate in an aqueous solution. According to one embodiment enema is a solution or a suspension. According to one embodiment, enema is a retention enema.

According to a preferred embodiment, the rectal dosage form is a rectal capsule.

According to one embodiment, the rectal dosage further comprises conventional excipients and carriers. Examples of excipients and carriers include, but are not limited to, texturizing agents, viscosity agents, solvents; adjuvants; lubricants; stabilizing agents; pH buffering agents; disintegrants; wetting agents; preservatives and the like.

According to one embodiment, the rectal dosage form comprises a viscosity agent. Examples of viscosity agents include, but are not limited to, polyvinylpyrrolidone, such as, for example, PVP K 90; cellulose derivatives, such as, for example, HPC H, HPC HF, HEC 250 HX and HEC 250 M; Lutrol F68; Ludvigel advanced; gums such as, for example guar gum, xanthan gum, such as Satiaxane UCX 930 or Satiagel USC 150, carraghenane gums and the like; carbopol derivatives, such as, for example, Carbopol 971P; silica derivatives and the like.

In one embodiment of the invention, the rectal dosage form comprises a viscosity agent, in an amount ranging from about 0.01 to about 50% in weight to the total weight of the rectal dosage form, preferably from about 0.01 to about 10% w/w, more preferably from about 0.4 to about 5.5% w/w. In one embodiment, the rectal dosage form comprises a viscosity agent in an amount ranging from about 0.5 to about 1% w/w, preferably from about 0.8 to about 0.95% w/w, more preferably of about 0.88% w/w. In another embodiment, the rectal dosage form comprises a viscosity agent in an amount ranging from about 1.5 to about 4% w/w, preferably from about 2 to about 2.5% w/w, more preferably of about 2.19% w/w. In another embodiment, the rectal dosage form comprises a viscosity agent in an amount ranging from about 4 to about 5.5% w/w, preferably from about 4.5 to about 5.4% w/w, more preferably of about 5% or about 5.25% w/w. In another embodiment, the rectal dosage form comprises a viscosity agent in an amount ranging from about 0.4 to about 1% w/w, preferably from about 0.45% to about 0.7% w/w, more preferably of about 0.47% or about 0.5% or about 0.67% w/w. In another embodiment, the rectal dosage from comprises a viscosity agent in an amount ranging from about 1.5 to about 5% w/w, preferably from about 2 to about 4% w/w, more preferably of about 4% w/w.

According to one embodiment, the rectal dosage form comprises a solvent. Examples of solvents include, but are not limited to, water such as, for example, purified water and UHQ water (wherein UHQ stands for Ultra-High Quality).

In one embodiment of the invention, the rectal dosage form comprises a solvent in an amount ranging from about 30% to about 99% in weight to the total weight of the rectal dosage form, preferably from about 35% to about 95% w/w, more preferably from about 40% to about 90% w/w. In one embodiment of the invention, the rectal dosage form comprises an amount of solvent ranging from about 80% w/w to about 85% w/w, preferably from about 82 w/w to about 83 w/w, more preferably is of about 82.25% w/w. In another embodiment of the invention, the rectal dosage form comprises an amount of solvent ranging from about 82.5 to about 87.5% w/w, preferably from about 85% w/w to about 86% w/w, more preferably of about 85.31% w/w. In another embodiment of the invention, the rectal dosage form comprises an amount of solvent ranging from about 85 to about 88% w/w, preferably from about 86% w/w to about 87% w/w, more preferably of about 86.63% w/w. In another embodiment of the invention, the rectal dosage form comprises a solvent in an amount ranging from about 40% to about 55% w/w, preferably from about 45% to about 50% w/w, more preferably of about 45% w/w, or about 49.5% w/w, or about 49.5% w/w. In another embodiment of the invention, the rectal dosage form comprises a solvent in an amount ranging from about 55 to about 75% w/w, preferably from about 60% to about 70% w/w, more preferably in an amount of about 66% or 66.2% w/w.

According to an embodiment, the rectal dosage form comprises from 1 mg to 800 mg of phenylbutyrate, preferably from 100 to 600, more preferably from 250 to 500. In one embodiment, the rectal dosage form comprises about 333.33 mg of phenylbutyrate. In another embodiment, the rectal dosage form comprises about 500 mg of phenylbutyrate. According to another embodiment, the rectal dosage form comprises an amount of phenylbutyrate ranging from about 1% w/w to about 80% w/w, in weight to the total weight of the rectal dosage form, preferably from about 5% w/w to about 75% w/w. In another embodiment, the amount of phenylbutyrate in the rectal dosage form ranges from about 10% w/w to about 15% w/w, preferably is of about 12.5% w/w. In another embodiment of the invention, the rectal dosage form comprises an amount of phenylbutyrate ranging from about 40% to about 55% w/w, preferably from about 45% to about 50% w/w, more preferably of about 45% w/w, or about 49.5% w/w, or about 49.5% w/w. In another embodiment of the invention, the rectal dosage form comprises an amount of phenylbutyrate ranging from about 55 to about 75% w/w, preferably from about 60% to about 70% w/w, more preferably in an amount of about 66% or 66.2% w/w.

According to one embodiment, the rectal dosage form comprises phenylbutyrate and excipients or carriers in a ratio in weight of phenylbutyrate:excipients/carriers ranging from about 1:1 to about 1:50, preferably from about 1:2 to about 1:25, more preferably from about 1:5 to about 1:10, and even more preferably of about 1:7. In another embodiment, the rectal dosage form comprises phenylbutyrate and excipients or carriers in a ratio in weight of phenylbutyrate:excipients/carriers ranging from about 1:0.1 to about 1:10, preferably from about 1:0.5 to about 1:3, more preferably from about 1:1 to about 1:2.

According to one embodiment of the invention, the pH of the rectal dosage form ranges from about 1 to about 12, preferably from about 5 to about 10, more preferably from about 7 to about 9.5. In one embodiment, the pH of the rectal dosage form ranges from about 7 to about 8.5, preferably from about 7.5 to about 8, more preferably is of about 7.79. In another embodiment, the pH of the rectal dosage form ranges from about 8.5 to about 9.5, more preferably from about 9 to about 9.25, more preferably is of about 9.14 or 9.19.

According to one embodiment of the invention, the density of the rectal dosage form ranges from about 0.5 to 1.5 g/ml, preferably from about 1 to about 1.2 g/ml, more preferably from about 1.05 to about 1.1. In one embodiment, the density of the rectal dosage form is of about 1.07 g/ml. In another embodiment, the density of the rectal dosage form is of about 1.08 g/ml. In another embodiment, the density of the rectal dosage form is of about 1.09 g/ml. The density of the rectal dosage form may be measured with a Brookfield apparatus.

According to one embodiment of the invention, the viscosity of the rectal dosage form ranges from about 1 to about 100 000 cP, preferably from about 10 to about 50 000 cP, more preferably from about 20 to about 12 000 cP. In one embodiment of the invention, the viscosity of the rectal dosage form ranges from about 20 to about 25 cP, preferably from about 21 to about 22 cP, more preferably is of about 21.4 cP. In another embodiment, the viscosity of the rectal dosage form ranges from about 1000 cP to about 3000 cP, preferably from about 1500 cP to about 2000 cP, more preferably is of about 1836 cP. In another embodiment, the viscosity of the rectal dosage form ranges from about 10 000 cP to about 12 000 cP, preferably from about 11 000 cP to about 11 800 cP, more preferably is of about 11 700 cP. In another embodiment of the invention, the viscosity of the rectal dosage form ranges from about 500 cP to about 5000 cP, more preferably from about 1000 cP to about 3500 cP, more preferably is of about 1370 cP, of about 1500 cP, of about 2010 cP or of about 3100 cP. The viscosity of the rectal dosage form may be measured with a Brookfield apparatus.

According to one embodiment, the dose administered is ranging from 1 to 600 mg/kg/day, preferably from 100 to 500 mg/kg/day. In one embodiment, the administered dose is of about 500 mg/kg of body weight/per day.

According to one embodiment, rectal administration of phenylbutyrate is performed in several days. According to an embodiment, rectal administration is performed from 1 to 10 times per day, preferably from 2 to 8 times per day, more preferably is performed 5 times a day. In one embodiment, the dose per administration ranges from about 1 to about 600 mg/kg of body weight, preferably from about 10 to about 500 mg/kg of body weight, more preferably from about 50 to about 200 mg/kg of body weight, and even more preferably is of about 100 mg/kg of body weight.

In one embodiment, the administered dose is of about 500 mg/kg of body weight/per day divided in 5 doses, i.e. each dose is of about 100 mg/kg of body weight/day.

The person skilled in the art knows how to adapt the dose to the condition of the patient. The dose is calculated per body weight of the patient in kg and per day.

According to one embodiment, the rectal dosage form is administered during at least one week, preferably at least two weeks, more preferably at least 4 weeks or more. According to another embodiment, the rectal dosage form is chronically administered, i.e. is administered for an unlimited period of time.

According to another embodiment, phenylbutyrate is rectally administered to maintain a high level of the drug in the patient. A person skilled in the art knows how to distribute the rectal doses over the day to achieve this objective.

According to one embodiment, the therapeutically acceptable salt of 4-phenylbutyrate is rectally administered to patients affected by a motor neuron disease, such as, for example, SMA; or by a metabolic disease, such as, for example, an urea cycle disorder or by cancer. According to an embodiment, patients affected by a motor neuron disease, such as, for example, SMA; or by a metabolic disease, such as, for example, an urea cycle disorder or by cancer are humans. According to another embodiment, patients are children, preferably aged between 0 and 5 years. According to a preferred embodiment, patients are infants, preferably aged between 0 and 18 months, more preferably between 0 and 12 months. According to another embodiment, the patients are aged from 5 years to 10 years. According to another embodiment, the patients are aged from 10 years to 18 years. According to another embodiment, patients are adults.

The present invention also relates to an acceptable salt of 4-phenylbutyrate as herein above described for treating a motor neuron disease or a metabolic disease or a cancer, wherein said acceptable salt of 4-phenylbutyrate is rectally administered.

The present invention also relates to a composition comprising an acceptable salt of 4-phenylbutyrate as herein above described for treating a motor neuron disease or a metabolic disease or a cancer, wherein said composition is rectally administered.

The present invention also relates to a pharmaceutical composition comprising an acceptable salt of 4-phenylbutyrate as herein above described and at least one pharmaceutically acceptable excipient, wherein said pharmaceutical composition is in a form suitable for rectal administration.

The present invention also relates to a medicament comprising an acceptable salt of 4-phenylbutyrate as herein above describes, wherein said medicament is in a form suitable for rectal administration.

### BRIEF DESCRIPTION OF THE FIGURES

FIG. 1: Plasma concentrations of 4-phenylbutyrate measured in treated animals administered via oral, IV or rectal routes.

### EXAMPLES

The present invention is further illustrated by the following examples that are not to be construed as limiting the scope thereof.

### Example 1: Rectal Dosage Forms of the Invention and Properties Thereof

### Formulations for Preclinical Studies (Formulations I, II and III)

Formulation I

| Ingredient | Function | Amount* |
|---|---|---|
| PVP K 90 (Polyvinylpyrrolidone) | viscosity agent | 5.25 |
| Purified water | Solvent | 82.25 |
| 4-phenylbutyrate | Active ingredient | 12.50 |

*amounts are in % in weight to the total weight of the rectal dosage form

Formulation II

| Ingredient | Function | Amount* |
|---|---|---|
| HPC H (Cellulose derivative) | viscosity agent | 2.19 |
| Purified water | Solvent | 85.31 |
| 4-phenylbutyrate | Active ingredient | 12.50 |

*amounts are in % in weight to the total weight of the rectal dosage form

Formulation III

| Ingredient | Function | Amount* |
|---|---|---|
| HEC 250 M (Cellulose derivative) | viscosity agent | 0.88 |
| Purified water | Solvent | 86.63 |
| 4-phenylbutyrate | Active ingredient | 12.50 |

*amounts are in % in weight to the total weight of the rectal dosage form

Properties of formulations I to III

| Formulation | Density (g/ml) | pH | Viscosity (cP) |
|---|---|---|---|
| I | 1.09 | 9.19 | 21.4 |
| II | 1.07 | 9.14 | 1836 |
| III | 1.08 | 7.79 | 11700 |

### Pediatric Formulations (Formulations IV to XI)

Formulation IV

| Ingredient | Function | Amount* |
|---|---|---|
| Carbopol 971P | Viscosity agent | 0.67 |
| Purified water | Solvent | 66 |
| 4-phenylbutyrate | Active ingredient | 33.33 |

*amounts are in % in weight to the total weight of the rectal dosage form

Formulation V

| Ingredient | Function | Amount* |
|---|---|---|
| PVP K 90 (Polyvinylpyrrolidone) | Viscosity agent | 3 |
| Purified water | Solvent | 47 |
| 4-phenylbutyrate | Active ingredient | 50 |

*amounts are in % in weight to the total weight of the rectal dosage form

Formulation VI

| Ingredient | Function | Amount* |
|---|---|---|
| HPC | Viscosity agent | 0.5 |
| Purified water | Solvent | 49.5 |
| 4-phenylbutyrate | Active ingredient | 50 |

*amounts are in % in weight to the total weight of the rectal dosage form

Formulation VII

| Ingredient | Function | Amount* |
|---|---|---|
| Lutrol F68 | Viscosity agent | 5 |
| Purified water | Solvent | 45 |
| 4-phenylbutyrate | Active ingredient | 50 |

*amounts are in % in weight to the total weight of the rectal dosage form

Formulation VIII

| Ingredient | Function | Amount* |
|---|---|---|
| HEC 250 HX | Viscosity agent | 0.5 |
| Purified water | Solvent | 49.5 |
| 4-phenylbutyrate | Active ingredient | 50 |

*amounts are in % in weight to the total weight of the rectal dosage form

Formulation IX

| Ingredient | Function | Amount* |
|---|---|---|
| Ludvigel Advanced | Viscosity agent | 0.47 |
| Purified water | Solvent | 66.2 |
| 4-phenylbutyrate | Active ingredient | 33.33 |

*amounts are in % in weight to the total weight of the rectal dosage form

Formulation X

| Ingredient | Function | Amount* |
|---|---|---|
| Satiaxane UCX 930 | Viscosity agent | 0.5 |
| Purified water | Solvent | 49.5 |
| 4-phenylbutyrate | Active ingredient | 50 |

*amounts are in % in weight to the total weight of the rectal dosage form

Formulation XI

| Ingredient | Function | Amount* |
|---|---|---|
| Satiagel USC 150 | Viscosity agent | 0.67 |
| Purified water | Solvent | 66 |
| 4-phenylbutyrate | Active ingredient | 33.33 |

*amounts are in % in weight to the total weight of the rectal dosage form

Properties of formulations IV to XI

| Formulation | pH | Viscosity (cP) |
|---|---|---|
| IV | 7.74 | N/A |
| V | 9.19 | 3100 |
| VI | 9.14 | 1500 |
| VII | 9.25 | N/A |
| VIII | 9.21 | 1370 |
| IX | 8.98 | N/A |
| X | 9.33 | 2010 |
| XI | 9.15 | N/A |

N/A: non-available

### Process for Preparing the Formulations of the Invention

The texturizing agent is dissolved in purified water in a glass beaker of 250 mL with a stirrer shaft with a deflocculating propeller. For a rapid solubilization, the stirring speed is maximal is reduced after 5 minutes for the removal of gas bubbles which may be introduced during the agitation. The pH of each solution obtained is measured before the addition of the active ingredient to observe its effect on the properties of the gelled solution.

Part of the solution is collected and put under slow stirring using a magnetic stirrer and a magnetic plate. To this fraction, the amount of active ingredient needed to obtain the desired final titration, with gradually increased stirring. Solution is let stand during 5 to 10 minutes, and then pH is measured.

Density of each solution is measured by taring, filling and weighing a 1 mL syringe.

### Example 2: Dissolution Rate of Rectal Capsules

Capsules in gelatin, size 3, are filled with sodium 4-phenylbutyrate with a manual filler. The dissolution rate is measured in different conditions of pH and temperature.

### Example 3: Evaluation of Pharmacokinetic

1. Evaluation of pharmacokinetic on a murine model.

These in vivo and analytical parts were conducted to estimate the levels of plasmatic concentrations and the pharmacokinetic (PK) parameters after a single administration of 4-Phenylbutyric Acid Sodium Salt by the oral, intravenous or rectal route to male Sprague Dawley rats.

### Materials and Methods

### Characteristics, Housing and Handling of Animals

31 male Sprague Dawley rats around 6 week old were used. These rats were supplied by the Elevage Janvier, France. On their arrival, the animals were numbered randomly and identified by an ear-tag. The health of animals was verified by observation. Animals were housed in makrolon cages with stainless steel wire lids with catches. The litter was supplied by U.A.R. (Epinay sur Orge, France) and was renewed at least every 72 hours. Temperature and humidity were continually monitored (Oceasoft® recording). The animal room conditions were kept as follows: Temperature: 22° C.±2° C. and Light/dark cycle: 12 h/12 h. Animals were fed on pellets (AO4C) supplied by U.A.R. (Epinay sur Orge, France). The pellets and tap water were given ad libitum. The animals were fasted over night before the administration.

### Treatment

For intravenous administration (IV), animals received 100 mg/kg body weight of 4-phenylbutyrate (corresponding to 1 ml/kg body weight of a solution at 100 mg/ml in UHQ water).

For oral administration (PO), animals received 500 mg/kg body weight of 4-phenylbutyrate (corresponding to 5 ml/kg body weight of tablets crushed and dissolved in UHQ water, wherein the final solution presents a concentration of 100 mg/ml).

For rectal administration, animals received 500 mg/kg body weight of 4-phenylbutyrate, corresponding to 3.7 ml/kg body weight of formulation I, II or III as described in Example 1, or to 3 ml of the formulation for intravenous administration

Formulations were kept under magnetic stirring during administrations.

### Sampling

| Route of | | Number of | |
|---|---|---|---|
| administration | Formulation | animals | Blood Sampling Time |
| IV | UHQ water | 3 | 5 min/30 min/2 h/4 h |
| | | 3 | 15 min/1 h/3 h/6 h |
| PO | Tablets to be | 3 | 5 min/30 min/2 h/4 h |
| | crushed | 3 | 15 min/1 h/3 h/6 h |
| Rectal | 1 | 3 | 5 min/30 min/2 h/4 h |
| | | 3 | 15 min/1 h/3 h/6 h |
| | 2 | 3 | 5 min/30 min/2 h/4 h |
| | | 3 | 15 min/1 h/3 h/6 h |
| | 3 | 3 | 5 min/30 min/2 h/4 h |
| | | 3 | 15 min/1 h/3 h/6 h |
| Rectal | Formulation IV | 1 | 15 min/1 h/3 h/6 h |

### Blood Sampling

Animals were anaesthetised with Isoflurane® using an anaesthetic system (Équipement Vétérinaire Minerve) during blood samplings. Blood was collected in retro-orbital sinus using a capillary tube. 0.3 to 0.4 ml of blood were collected per time-point. Lithium heparin was used as anticoagulant.

Blood samples were centrifuged at 2500 rpm at +4° C. (between 0 and 9° C.), the plasma was removed and placed into labelled polypropylene tubes. Individual plasma samples were stored frozen (−20° C.±5° C.) until analyses.

### Analytical Method

Diluted solutions of 4-Phenylbutyric Acid Sodium Salt and of Phenylacetate (metabolite of phenylbutyrate) was performed in UHQ water, in order to obtain final concentrations of calibration standards in blank rat plasma at 1, 5, 10, 25, 50, 100, 500, 1000 and 2000 ng/mL.

Extraction was performed by protein precipitation. 300 μL of acetonitril was added to 100 μL of plasma samples, calibrations standard and S0. The mixtures were centrifuged at 3500 rpm, during 5 minutes at +4° C. (between 0 and 9° C.), supernatants were removed and placed into labelled polypropylene vials or plates. Extracts were stored between 0 and 9° C. until injection into the LC-MS/MS system (LC-MS/MS stands for Liquid chromatography coupled to tandem mass spectrometry).

A calibration curve was run before and after samples in order to mean the possible signal deviation. Concentrations were calculated according to the mean of both calibration curves.

The criteria thereafter will be used to validate the calibration curves: (1) deviation from the nominal value between −30.0% and +30.0% and (2) calibration standards were excluded if they did not be ranged between −30.0% and +30.0%.

### Concentrations Determination

Concentrations of the samples were calculated directly after automatic integration by Analyst® 1.5.1. They were expressed in μg/mL for the plasma.

Mean plasma concentration of the substance was calculated using individual concentration and was expressed with the corresponding standard deviation value and variation coefficient (with CV(%)=SD/Concentration mean×100).

### Pharmacokinetic Analysis

The pharmacokinetic analysis was carried out using KINETICA® (Version 4.3—Thermo Electron Corporation—Philadelphia—USA). An independent model (non compartmental analysis) was used.

### Results

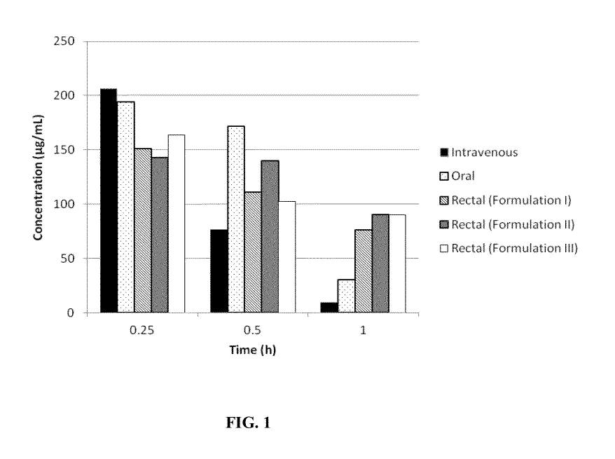

Plasma concentrations of 4-phenylbutyrate measured in treated animals are shown in FIG. 1. As shown in FIG. 1, plasma concentrations of 4-phenylbutyrate measured 1 hour after rectal administration are about 5 to 10 folds more elevated than after intravenous administration and about 2 to 3 folds more elevated than after oral administration of 4-phenylbutyrate.

Same results were obtained for the plasmatic concentration of Phenylacetate.

No significant difference in plasma concentrations of 4-phenylbutyrate according to the rectal formulation (I, II or III) was detected.

2. Evaluation of pharmacokinetic on a juvenile minipig model.

The aim of this study was to test the toxicity of repeated administrations of sodium phenylbutyrate (NaPB) by the intended human route in juvenile minipigs (13-week study, total dose divided into 5 doses per day). Minipig is a model of interest for studies of local tolerance due to similarities between properties of the skin of pigs of the one of humans.

### Materials and Methods

A 13-week repeat-dose toxicity study was conducted in juvenile minipigs using the rectal administration route of sodium phenylbutyrate (NaPB). One group was treated orally at the pharmacological dose of NaPB. One group received oral NaPB (total dose divided into 5 doses per day) for comparison.

The study included safety pharmacology evaluation, toxicokinetic (TK) determination, as well as a four-week recovery period.

Earliest dosing was started at 7 days of age. Oral gavage (5 times daily at 3-4 hour intervals) and rectal administration (5 times daily at 3-4 hour intervals) were performed during 13 weeks. A recovery period of 28 days was performed.

The piglets were divided into 5 groups (2 sows per group) as shown in Table below:

| | Main animals | Recovery animals |
|---|---|---|
| | (Males + Females) | (Males + Females) |
| Rectal administration, control | 3 + 3 | 2 + 2 |
| Rectal administration, low | 3 + 3 | |
| Rectal administration, | 3 + 3 | |
| medium | | |
| Rectal administration, high | 3 + 3 | 2 + 2 |
| Oral administration, low | 3 + 3 | |

The doses were selected as follows:
Low = the pharmacological dose (550 mg/kg);
Medium = a multiple of the low dose (5x, i.e., 2,750 mg/kg);
High = a multiple of the medium dose (5x, i.e., 13,750 mg/kg).

The recovery animals were housed for additional 28 days after dosing. The piglets were weighed twice weekly throughout the study. Growth measurements were performed twice weekly. Ophthalmology, electrocardiography were done the last day of treatment. For the clinical pathology, hematology and blood chemistry analyses were performed on all piglets before necropsy. The parameters measured met standard guidelines. For the toxicokinetics, a single blood sample was taken from the precaval sinus of each piglet on the first day of dosing (4 timepoints, 1 animal per sex/time point). In addition blood samples were collected from all piglets at 4 time points on the last day of dosing (4 animals/sex/timepoint).

### Results

No toxicity was observed, whatever the rectal dose of NaPB administered.

No significant difference according to the sex of the animal was recorded.

The pharmacokinetic properties of rectally administered NaPB were found to be increased as compared to oral administration. Indeed, blood concentrations of the combination of phenylbutyrate and phenylacetate (a metabolite of phenylbutyrate) measured 2 hours after rectal administration were about 1.5 times higher than the concentrations measured after oral administration of the same amount of NaPB.

### Example 4: Efficacy

The efficacy of the treatment was evaluated in a murine model for SMA. One test consisted in the dosage of the SMN protein in different tissues such as blood, muscles, liver, brain or spinal cord.

Parameters such as weight, temperature, general aspect, vitality, length of life were also evaluated at different ages, as well as the effect of phenylbutyrate on vital function, especially on respiration and heart rate.

Motor assessment was also tested to determine the efficacy of the treatment. The sensorimotor development was evaluated measuring the traveled distance, orientation, speed, number of rotation and paths in straight line.

Results showed that mice receiving the rectal dosage form of the invention present an increased length of life as compared to non-treated mice.

### Example 5: Phase I Clinical Trial

A phase 1 cross-over pharmacokinetic study will be performed in adult healthy volunteers with NaPB administered orally and rectally to assess the local safety and the pharmacokinetics profiles of the medicinal product provided via the two different routes of administration.

The synopsis of the Phase 1 protocol is provided in the Table below.

| | |
|---|---|
| Clinical Trial Phase | Phase 1 |
| Study Site | Monocentric site |
| Study Period | 2 days of treatment (D1 and D2) orally (daily dose divided |
| | into 5 administrations), three days of washout (D3, D4, and |
| | D5, and 2 days of treatment (D6 and D7) via the rectal route |
| | (daily dose divided into 5 administrations). |
| Study Population | Male and Female Healthy Volunteers |
| Primary Study | To evaluate the pharmacokinetic (PK) parameters of oral and |
| Objective | rectal sodium phenylbutyrate (NaPB) in healthy volunteers in |
| | order to propose an adequate rectal formulation and strength to |
| | treat infants suffering from spinal muscular atrophy in a |
| | subsequent pivotal clinical study. |
| Secondary Study | To compare the safety and tolerability of NaPB administered |
| Objectives | via the oral and rectal routes. |
| Study Design | This is a mono-center cross-over, placebo controlled, phase 1 |
| | study in adult healthy volunteers. |
| | It is planned that 10 volunteers in total will be enrolled in the |
| | study. |
| | Eight (8) subjects will receive oral NaPB (500 mg/kg/day |
| | divided into 5 doses, i.e., 100 mg/kg/administration) during 2 |
| | days. Blood samples for PK determination will be taken after |
| | the first and the 10th administration. Safety assessments will |
| | be performed during the treatment period. |
| | After a 3-day washout period, the same 8 subjects will receive |
| | rectal NaPB (500 mg/kg/day divided into 5 doses, i.e., 100 mg/kg/ |
| | administration) for two days. Blood samples for PK |
| | determination will be taken after the first and the 10th |
| | administration. Safety assessments will be performed during |
| | the treatment period. |
| | Two (2) subjects will receive oral placebo divided in 5 |
| | administrations per day during 2 days, and then after the 3-day |
| | washout period rectal placebo 5 times a day during two days. |
| Sample Size | 10 volunteers (4M + 4F on active for both oral and rectal |
| | administrations, 1M + 1F on placebo for both oral and rectal |
| | administrations). |
| Inclusion Criteria | Classical inclusion criteria for healthy volunteers. |
| Exclusion Criteria | Classical exclusion criteria for healthy volunteers |
| Primary Outcome | The plasmatic levels of NaPB (and the metabolite |
| Measure | phenylacetate) will be determined. The excretion of the later in |
| | the urine will also be detected in the form of |
| | phenacetylglutamine. PK analyses will be performed for the |
| | 1st and the 10th oral and rectal administrations of NaPB (4 PK |
| | curves will be determined). The timing of blood withdrawal |
| | remains to be determined. |
| | The relative bioavailability for each subject and for both |
| | routes will be determined, as well as the average plasma |
| | concentrations vs. time. Then the AUCs obtained with the two |
| | routes will be compared. |
| Secondary Outcome | The safety and tolerability will be determined by the reporting |
| Measures | of adverse events and serious adverse events, and physical |
| | examination (including vital signs measurements). |

1-15. (canceled)

16. A method for treating a motor neuron disease or a metabolic disease in a subject in need thereof, comprising rectally administering a therapeutically acceptable salt of 4-phenylbutyrate to the subject.

17. The method according to claim 16, wherein the therapeutically acceptable salt of 4-phenylbutyrate is administered at a dose ranging from 1 to 600 mg/kg/day.

18. The method according to claim 16, wherein the therapeutically acceptable salt of 4-phenylbutyrate is administered from 1 to 10 time per day.

19. The method according to claim 16, wherein the subject is a child suffering from a motor neuron disease or a metabolic disease.

20. The method according to claim 16, wherein the subject is a 0-5 years child suffering from a motor neuron disease or a metabolic disease.

21. The method according to claim 16, wherein the subject is an infant suffering from a motor neuron disease or a metabolic disease.

22. The method according to claim 16, wherein said motor neuron disease is selected from the group comprising spinal muscular atrophy, amyotrophic lateral sclerosis, ischemia, thalassemias, cystic fibrosis, and Huntington disease.

23. The method according to claim 16, wherein said motor neuron disease is spinal muscular atrophy.

24. The method according to claim 16, wherein said metabolic disease is selected from the group comprising urea cycle disorder and maple syrup urine disease.

25. A composition comprising a therapeutically acceptable salt of 4-phenylbutyrate in a rectal dosage form.

26. The composition according to claim 25, wherein the therapeutically acceptable salt of 4-phenylbutyrate is selected from the group comprising sodium, potassium, magnesium or calcium salt.

27. The composition according to claim 25, wherein the salt is the sodium salt.

28. The composition according to claim 25, wherein the rectal dosage form is selected from the group comprising suppositories, rectal capsules, enemas, rectal gels, rectal foams or rectal ointments.

29. The composition according to claim 25, comprising from 1 to 800 mg of a therapeutically acceptable salt of 4-phenylbutyrate.

30. The composition according to claim 25, comprising at least one viscosity agent, and a solvent.

31. The composition according to claim 25, comprising at least one viscosity agent and a solvent, wherein said viscosity agent is selected in the group consisting of polyvinylpyrrolidone, cellulose derivatives, gums, and carbopol derivatives.

32. The composition according to claim 25, comprising at least one viscosity agent and a solvent, wherein said viscosity agent is selected in the group consisting of PVP K 90, HPC H, HPC HF, HEC 250 HX, HEC 250 M, xanthan gum, and Carbopol 971P.
